(19) United States
(12) Patent Application Publication
(10) Pub. No.: US 2021/0147805 A1
(43) Pub. Date: May 20, 2021

(54) IN VITRO METHOD FOR PROVIDING STEM CELL DERIVED CARDIOMYOCYTES

(71) Applicant: NCARDIA B.V., Leiden (NL)

(72) Inventors: Benjamin WOLTERS, Köln (DE); Ralf KETTENHOFEN, Köln (DE); Heribert BOHLEN, Köln (DE); Stefan Robbert BRAAM, Leiden (NL)

(21) Appl. No.: 16/649,475

(22) PCT Filed: Sep. 21, 2018
(86) PCT No.: PCT/NL2018/050627

§ 371 Date: Mar. 20, 2020

(30) Foreign Application Priority Data
Sep. 22, 2017 (NL) 2019618

(51) Int. Cl. C12N 5/077; G01N 33/50; C12N 15/87

(52) U.S. Cl. CPC C12N 5/0657; G01N 33/5014; G01N 33/5073; C12N 2529/10; C12N 2506/45; C12N 2510/02; C12N 15/87

(57) ABSTRACT

The current invention relates to finding that stem cell derived, for example induced pluripotent stem cell derived, cardiomyocytes with both a matured electrophysiological phenotype and optical excitability for high-fidelity beating frequency modulation may be obtained. With the method of the invention stem cell derived cardiomyocytes may obtained that show a mature phenotype in comparison to the same stem cell derived cardiomyocytes not be subjected to the method of the invention disclosed herein, the latter displaying an immature phenotype.

### BACKGROUND OF THE INVENTION

Progress in several areas of medicine (i.e. mental health, cardiology, immunity etc.), where new and more effective drugs are needed, is severely impeded by the extreme cost engendered by undesired drug-induced adverse effects associated with several candidate lead compounds in the drug discovery pipeline. One major drawback of the current drug discovery process, for all drug development, is the high rate of attrition of lead compounds caused by unforeseen adverse drug effects, notably cardiotoxicity, often detected in the later rather than in the earlier phases of the drug discovery pipeline.

The prevention of drug-induced cardiotoxicity, which may manifest itself as cardiac arrhythmias, represents the highest priority for regulatory agencies and (bio)pharmaceutical companies, since manifestation of this type of toxicity is immediately life-threatening. It has been shown that 33% of adverse safety events in clinical studies are generally attributed to cardiac arrhythmic effects, which may lead to sudden death or severe cardiac complications in subjects (Mordwinkin et al (2013) Journal of cardiovascular translational research, Vol: 6(1):22-30).

Therefore, there is an urgent need for new cost-effective strategies to improve the traditional process of drug development/discovery, e.g. eliminate drug-induced cardiotoxicity and identify novel efficacious drugs and/or compounds to treat cardiac diseases. Particularly, there is an urgent unmet need for predictive human disease models for various stages of drug discovery and development.

Over the last decade, considerable research efforts have been devoted towards this goal. For instance, several biological models and tools have been developed including the use of pluripotent stem cells, ion-channel assays, and computational tools. Particularly, the use of cardiomyocytes derived from pluripotent stem cells is a current focus of interest in the development of innovative predictive assays in drug discovery and development.

Pluripotent stem cells are a potential source of cells for generating cardiomyocytes in in vitro culture. The use of cardiomyocytes is not only important for the development of assays for predicting drug-induced toxicity for all drugs in development, but is also important for cardiac research as well as for the development of new cardiac drugs in general, where cardiomyocytes can be used to uncover new drug targets and assess cardiac drug safety.

The ability to use cardiomyocytes in drug development/discovery, drug safety assay, cardiac disease modelling, cardiac research, and other biological purposes largely depends on the ability to cultivate and obtain cardiomyocytes derived from stem cells in culture (in vitro), which must meet certain phenotypic requirements such as, for instance, a certain level of functional properties (e.g. adult-like electrophysiological patterns) and/or genetic profile (e.g. adult-like expression of cellular fate-specific genetic markers), and/or morphological aspects. (e.g. adult-like shape and histological properties).

In this respect, a main limitation of the art is that current methods for generating cardiomyocytes in in vitro culture or in vitro culture system yield results which are suboptimal and do not or only in part match the requirements for applications such as drug development/discovery, drug safety assay, cardiac research, regenerative medicine, where cardiomyocytes suitable for ‘real-life’ context (i.e. adult-like state) especially with respect to functionality are needed.

That is because current methods yield immature ventricular and/or atrial cardiomyocytes, which are akin to fetal (fetal-like) cardiomyocytes. Such cardiomyocytes are inadequate or not optimal for use in applications such as drug development/discovery, drug safety assay, cardiac disease modelling, cardiac research, and other purposes aimed to model or treat cardiac conditions that typically occur in adulthood and not at earlier developmental stages. Hence, adult-like (mature) cardiomyocytes would be better suited than immature cardiomyocytes or fetal-like cardiomyocytes for these purposes.

In addition, the cell autonomous spontaneous beating is a source of variation in assays. It is impossible to pace frequencies that are lower than the spontaneous beating rate and the lack of control in this respect makes it difficult to test compounds at various frequencies. This is desired to get a full physiological understanding of the compound to be tested and to test parameters like use dependency and reverse use dependency. Currently there is no (reliable) method to overcome these issues.

Therefore, there is a need for improved methods for generating assay systems consisting of adult-like cardiomyocytes (as opposed to immature cardiomyocytes) from stem cells like pluripotent (embryonic) stem cells and/or induced pluripotent (stem) cells in in vitro culture.

It is an object of the present invention to overcome one or more major limitations of the art by providing a new and inventive in vitro method for providing stem cell derived cardiomyocytes with advantageous characteristics and the use of these cardiomyocytes in assay systems, for the above-identified uses.

### DESCRIPTION

### Definitions

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular forms “a,” “an” and “the” include plural referents unless the context clearly dictates otherwise. For example, a method for administrating a drug includes the administrating of a plurality of molecules (e.g. 10's, 100's, 1000's, 10's of thousands, 100's of thousands, millions, or more molecules).

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “and/or” indicates that one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

As used herein, “to comprise” and its conjugations is used in its non-limiting sense to mean that items following the word are included, but items not specifically mentioned are not excluded. It also encompasses the more limiting “to consist of”.”

As used herein, the term “stem cell” is well-known to the skilled person and refers to undifferentiated cells defined by their ability at the single cell level to both self-renew and differentiate to produce progeny cells, including self-renewing progenitors, non-renewing progenitors, and terminally differentiated cells. Stem cells have the ability to divide for indefinite periods in culture. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts. Stem cells are categorized as somatic (adult) stem cells or embryonic stem cells and may be multi, pluri or omnipotent in nature. A somatic stem cell is an undifferentiated cell found in a differentiated tissue that can renew itself (clonal) and (with certain limitations) differentiate to yield all the specialized cell types of the tissue from which it originated. With the context of the current invention the stem cell may, for example be both a somatic stem cell or an embryonic stem cell.

As used herein, the term “embryonic stem cells”, in the art also abbreviated as ‘ES cells’ or ESC (or if of human origin ‘hES cells’ or ‘hESCs’) as used herein is generally understood by the skilled person and refers to pluripotent embryonic stem cells (PESO) that are derived from the inner cell mass of a blastocyst. The skilled person understands how to obtain such embryonic stem cells, for example as described by Chung (Chung et al (2008) Stem Cell Lines, Vol 2(2):113-117), which employs a technique that does not cause the destruction of the donor embryo(s). PESO can differentiate into cells derived from any of the three germ layers. PESO can be distinguished from other types of cells by the use of markers or lineage-specific markers including, but not limited to, Oct-4, Nanog, GCTM-2, SSEA3, and SSEA4.

As used herein, “pluripotent stem cells” are stem cells capable of self-replication that have an ability to differentiate at least into one type each of differentiated cells that belong to ectoderm (e.g. interior stomach lining, gastrointestinal tract, the lungs), mesoderm (e.g. heart, muscle, bone, blood, urogenital tract), and endoderm (e.g. epidermal tissues and nervous system). Examples of pluripotent stem cells include an induced pluripotent stem cell (iPS cell), an embryonic stem cell (ES cell), an embryonic germ cell (EG cell), an embryonal carcinoma cell (EC cell), a multipotent adult progenitor cell (MAP cell), and an adult pluripotent stem cell (APS cell). Also included in this definition are transdifferentiated cells, for example form fibroblast to cardiomyocytes (see, e.g. Okada, pp. 349-380, In: Current Topics in Developmental Biology, Denis-Donini et al. eds., Acad. Press, 1980; Okada, Trans-differentiation, Oxford Sci. Publ., 1991 and/or U.S. Pat. No. 6,670,397 B1). The term “pluripotency” is generally understood by the skilled person as referring to this attribute of the pluripotent stem cell that to differentiate.

As used herein, the term “induced pluripotent stem cells”, commonly abbreviated as iPSC or iPS cell, refers to somatic (adult) cells reprogrammed to enter an embryonic stem cell-like state by being forced to express factors important for maintaining the “sternness” of embryonic stem cells. Typically, iPSC are artificially prepared from a non-pluripotent cell, (i.e. adult somatic cell, or terminally differentiated cell) such as fibroblast, a hematopoietic cell, a myocyte, a neuron, an epidermal cell, or the like, by introducing into or otherwise contacting the cell with reprogramming factors. The term “induced pluripotent stem cells (iPSC)” does not include embryonic stem cells.

As used herein, the term tardiomyocytes' or ‘cardiac myocytes’ as used herein refers generally to any cardiomyocyte lineage cells, and can be taken to apply to cells at any stage of cardiomyocyte ontogeny, unless otherwise specified. For example, cardiomyocytes may include both cardiomyocyte precursor cells (immature cardiomyocytes or fetal cardiomyocytes) and mature cardiomyocytes (adult-like cardiomyocytes). Further, cardiomyocytes can be subdivided into subtypes including, but not limited to, atrial cardiomyocyte, ventricular cardiomyocyte, sinoatrial node (SA) nodal cardiomyocyte, peripheral SA nodal cardiomyocyte, or central SA nodal cardiomyocyte. According to the current invention, the cardiomyocytes are obtained by differentiation (and maturation) of stem cells by in vitro culturing techniques.

For example, cardiomyocytes may include both cardiomyocyte precursor cells and mature cardiomyocytes. The term “stem cell-derived cardiomyocytes” as used herein refers to cardiomyocytes that are generated from pluripotent stem cells, for examples from human pluripotent stem cells such as adult stem cells, human embryonic stem cells or human induced pluripotent stem cells, by the process of differentiation, or by the process of trans differentiation.

In particular, within the context of the current invention, the term “stem cell-derived cardiomyocytes” refers to such cardiomyocytes obtained by in vitro culturing, for example by in vitro differentiation of (human) pluripotent stem cells. Such stem cell-derived cardiomyocytes can be defined as spontaneously contractile cells derived by in vitro methods from a human pluripotent cell, although sometimes non-contractile cells can be obtained.

Recent reviews defining and describing stem-cell derived cardiomyocytes, within the context of the current invention, have covered methods to create (e.g. Vidarsson et al. Stem Cell Rev. 2010; 6(1):108-120, Boheler et al. Circ Res. 2002; 91(3):189-201. Mummery et al. Circ Res. 2012; 111(3):344-358, and Jiang et al. J Cell Mol Med. 2012; 16(8):1663-1668, David et al. Physiology (Bethesda) 2012; 27(3):119-129), and purify (Habib et al. J Mol Cell Cardiol. 2008; 45(4):462-474) such stem-cell derived cardiomyocytes, as well as their electrophysiology (Blazeski et al. Prog Biophys Mol Biol. 2012; 110(2):178-195), and these methods and media, for example based on APEL (StemCell Technologies) and StemPro34 (Invitrogen), used are well known to the skilled person.

Cardiomyocytes directly obtained from an adult or mature heart, e.g. human heart, are understood to not be “stem cell-derived cardiomyocytes” within the context of the current disclosure. Whereas cardiomyocytes derived (in vitro) from a human heart that are cardiac progenitor cells or cardiac stem cells (or trans-differentiated from cardiac or dermal fibroblast cells) may be considered stem cell-derived cardiomyocytes within the context of the current disclosure

The term ‘fetal-like cardiomyocytes’ or ‘immature cardiomyocytes’ as used herein refers to a cardiomyocyte derived from stem cells in in vitro culture, which does not possess the desired phenotype and/or genotype in relation to an adult or adult-like cardiomyocyte. For instance, such fetal-like (immature) cardiomyocytes may exhibit automaticity (spontaneous contraction) and/or fetal-type ion channel expression, and/or fetal-type electrophysiological signals, and/or fetal-like gene expression patterns, and/or fetal-type physical phenotypes. Fetal-like (immature) cardiomyocytes may, for example, be distinguished from other cell types by using markers or lineage-specific markers including, but not limited to, MYH6, TNNT2, TNNI3, MLC2V, EMILIN2, SIRPA, VECAM, and others markers suitable for assessing a fetal or fetal-like stage of development (Burridge et al (2012) Stem Cell Cell, Vol. 10(1):16-28). The metabolic maturity can be determined by methods known to the skilled person, for examples methods that look at one of more of phenotype, morphology, gene expression, metabolic markers, cell surface markers, electrophysiological characteristics and/or cellular functional assay of the cell. For example, for maturation one can determine decreased expression of genes associated with a “fetal” state or cardiac hypertrophyic state such as, for example, NPPA (BNA) and NPPB (BNP), or preferably, determine the electrophysiological characteristics of the maturing stem-cell derived cardiomyocytes, and wherein a more adult or adult-like cardiomyocyte characteristic can be seen for more maturated stem-cell derived cardiomyocytes, as discussed in detail herein.

In addition, the relative maturity of stem-cell derived cardiomyocytes can be determined by the presence of decreased expression of genes associated with a fetal state, such as NPPA, NPPB, smooth muscle actin and skeletal actin, or the increasing expression of adult genes/proteins, such as myosin light chain 2V, calsequestrin and ryanodine receptor), but is not limited to those as other means of determining are known to the skilled person.

The term ‘adult-like cardiomyocytes’ or ‘mature cardiomyocytes’ as used herein refers to cardiomyocytes which possess the desired phenotype and/or genotype in relation to an adult cardiomyocyte. In one embodiment, a mature cell has the phenotype and/or genotype of, but is not limited to, an adult cardiomyocyte or atrial cardiomyocyte or ventricular cardiomyocyte or SA nodal cardiomyocyte or peripheral SA nodal cardiomyocyte or central SA nodal cardiomyocyte.

In other embodiments, adult-like cardiomyocytes' or ‘mature cardiomyocytes’ exhibit more mature electrophysiology patterns and/or more mature calcium handling patterns, and/or more adult-type ion channel expression, and/or more adult-type electrophysiological signals, and/or more adult-like contractile properties, and/or more adult-like gene expression patterns, and/or more adult-type physical (morphological) phenotypes when compared to fetal-like (immature) cardiomyocytes derived from stem cells by in vitro culture. Adult-like cardiomyocytes may also harbor greater degree of myofibril organization and sarcomeric striations, which are features that are poorly or insufficiently developed in the fetal-like (immature) cardiomyocytes derived from stem cells in in vitro culture.

The skilled person knows how to assess the maturity of stem cell derived cardiomyocytes in an in vitro culture, for example by using a set of known cardiomyocyte-specific markers or lineage-specific markers relevant for a particular developmental stage as well as available methods in the art so as to distinguish adult-like (mature) cardiomyocytes from fetal-like (immature) cardiomyocytes. In the present invention, one way to assess maturity of stem cell derived cardiomyocyte in in vitro culture is to assess the expression of gene markers associated with the fetal (immature) state such as (but not limited to) NPPA, NPPB, Ryr2, smooth muscle actin, and skeletal actin. Decreased (gene) expression of one or more of said fetal (immature) markers would be indicative of an adult-like (mature) state.

Another way to assess maturity of stem cell derived cardiomyocytes in an in vitro culture is to assess their (electro)physiological properties, for instance the ability of a cell to generate and/or propagate an action potential in vitro. Electrophysiological maturity of stem cell derived cardiomyocytes in in vitro culture can be for instance assessed by patch clamp techniques, among other techniques. Changes that are indicative of an adult-like (mature) electrophysiological phenotype, in comparison to an fetal-like (immature) electrophysiological phenotype, include (but are not limited to) increased maximum upstroke velocity, decreased resting membrane potential, and increased amplitude of the action potential, which are hallmarks of an adult cardiomyocyte. Physiological changes are increase in contraction force.

Another way to assess maturity of stem cell derived cardiomyocytes in an in vitro culture is to assess their morphological features, for instance the shape and/or ultra-structural organization of the cytoskeleton.

As used herein, the term “differentiation”, refers to a process of progressive transformation by which a cell acquires biochemical and morphological properties required to perform a specialized function. The differentiation level, as used herein, refers to an extent of progression of differentiation from stem cell to a cell having no ability for differentiation, that is a terminally differentiated cell. Although such a differentiation level varies for each cell a person skilled in the art can appropriately decide the criteria in accordance with the application purpose or the like. Stem cell derived cardiomyocytes may thus refer to a cells that have differentiated, by in vitro culturing, from stem cells into cells that have acquired biochemical and morphological properties characteristic for a—in vitro cultivated—cardiomyocytes. An undifferentiated cell or dedifferentiated cell thus refers to a cell with a lower differentiation level, that is, a cell with higher ability for differentiation, as compared to a differentiated cell, that refers to a cell with a higher differentiation level, that is, a cell with lower ability for differentiation. Dedifferentiation may for example refer to any of the loss of contractility apparatus, calcium handling capabilities, maintenance of transmembrane voltage potential, or the gain of the ability to divide or produce progeny above the rate usually observed in mature adult like cardiomyocytes.

As used herein, ‘maturation’ refers to a biological process wherein a differentiated cell derived from stem cells exhibiting an immature state (or fetal state) of development is allowed to attain an (even) more functional or a fully functional (including genetic and morphological) state of development which is usually post mitotic in nature under controlled conditions in anin vitro culture.

As used herein, “mammal” refers to any member of the class Mammalia, including, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term. Preferably the mammal is human.

The term “pacing” as used herein refers to a situation in which a cell, e.g. cardiomyocytes in culture, is stimulated at a certain frequency. The purpose of pacing is to induce in vitro cardiomyocytes to beat synchronously and at a higher beat frequency, e.g. to achieve a beat rate that is closer to physiological level. Pacing, as described herein, also reduces variations in the beat rate of in vitro cardiomyocytes across wells (e.g. MEA wells), assays, treatment, etc. Pacing, as described herein and within the method as disclosed, advantageously allows:

1. To control as well as study beat rate-induced effects on drug action in in vitro cardiomyocytes. Beat rate-induced effects on drug actions are often referred to as “use-dependent” as well as “reverse use-dependent” drug actions on cardiac ion channels.

The term “use-dependent effect” or “use-dependence” as used herein refers to a situation wherein a drug or compound binds to its target channels more avidly or effectively when the channel is stimulated at a relatively fast beat rate. In other words, compounds associated with “use-dependent effect” can exert more potent effects or actions on cardiomyocytes when the cardiomyocytes (e.g. in vitro ventricular cardiomyocytes) have a relatively fast beat rate (Grant, A (2009) in Circ. Arrhythmia Electrophysiol, Vol. 2, pages 185-194).

The term “reverse use-dependent” or “reverse use-dependence” as used herein refers to a situation wherein a drug or compound binds to its target channels more avidly or more effectively when the channel is stimulated at a relatively slow or low beat rates. In other words, compounds associated with “reverse use-dependent effects” can exert more potent effects or actions on cardiomyocytes when the cardiomyocytes (e.g. in vitro ventricular cardiomyocytes) have a relatively low or slow beat rate (Hondeghem and Snyders (1990) in Circulation, Vol 81, pages 686-690).

2. To control as well as study drug-induced effects on the beat rate of in vitro cardiomyocytes (e.g. in vitro ventricular cardiomyocytes). For instance, pacing, in the context of the present invention, can also be used to control and/or study drugs or compounds that can exert direct or indirect effects on the beat rate of in vitro cardiomyocytes irrespective of the beat rate per se (i.e. not associated with (reverse) use-dependent effects as discussed herein). In this context, pacing can be advantageously used to control and/or study dose-dependent effects of compounds.

The term “optogenetic pacing” or “pacing” as used herein refers to a pacing technique which relies on the use of genetically targeted, light-activated proteins, such as Channel Rhodopsin 2 (ChR2), to remotely and dynamically alter (e.g. “pace”) the activities of excitable cells (e.g. in vitro ventricular cardiomyocytes). Instead of using electrical stimulation, optogenetic pacing technology relies on the use of light (e.g. blue light (475 nm)) as a pacing stimulus to pace cells. For instance, when a cell (e.g. in vitro ventricular cardiomyocytes) expressing ChR2 or genetically manipulated to express ChR2 (e.g. in vitro ventricular cardiomyocytes expressing ChR2) is activated by blue light (475 nm), ChR2 will cause the influx of cationic ions, mainly sodium ions (Na+), which will consequentially depolarize the membrane potential and evoke an action potential leading to the expression of a cardiac beat (Bruegmann et al (2010) Nature Methods, Vol. 7, pages 897-900).

Optogenetic pacing can be performed at various frequencies, e.g. by varying the number of flash of lights per minute and/or duration of the flash of light stimulus per minute and/or intensity of the light per minute. Optogenetic pacing using various frequencies can also be performed in an incremental manner as taught herein. In other words, by varying the number of flash of lights per minute and/or duration of the flash of light stimulus per minute and/or intensity of the light per minute, the beat rate of in vitro cardiomyocytes (e.g. in vitro ventricular cardiomyocytes) can be altered or changed, e.g. be more synchronized, less variable, and gradually increased towards reaching a maximal beat rate or beat frequency that is closest to the absolute maximum that would be achievable in the in vitro ventricular cardiomyocytes under study.

In the context of the present invention, the in vitro cardiomyocytes, e.g. in vitro ventricular cardiomyocytes may be modified to express a suitable light-activated protein (preferably being also modified to express a Kir2.x channel), such as Channel Rhodopsin 2 (ChR2), for the purpose of performing in vitro optogenetic pacing using any suitable methods.

The term “microelectrode array” (abbreviated MEA) or “microelectrode array system” as used herein refers to a technology platform used to measure electrical activity from cells (e.g. cardiomyocytes) in culture. MEA system consists of electrodes embedded in a cell culture substrate in a manner that allows the electrode to interface with established cardiomyocyte networks in a non-invasive manner so as to provide functional, mechanistically-based measures of the cardiac action potential without perturbing the cellular network. The electrophysiological signal obtained from the microelectrodes, termed the extracellular field potential (FP), arises from the propagation of the cardiac action potential (AP) across the electrode array, providing measures of the depolarization and repolarization of the cardiomyocyte network (e.g. in vitro monolayer of ventricular cardiomyocytes, which are electrically connected) that are directly correlated to corresponding measures from the cardiac action potential waveform. Existing commercial MEA systems provide direct voltage recordings of high spatial and temporal resolution across multiple wells simultaneously, providing an effective and efficient platform technology for evaluation of cardiomyocyte activity. Non-limiting examples of MEA systems include Axion Maestro MEA multi Chanel System from Axion Biosystems, Atlanta, Ga. USA; ACEA xCELLigen RTCA CardioECR from ACEA Biosciences, Inc San Diego, Calif. USA; Nanion CardioExcytes 96 from Nanion technologies Munich Germany; Hamamatsu FDSS/microcell kinetic plate reader from Hamamatsu Almere, Netherlands, and the like.

The terms “encodes”, “encoding”, “coding sequence”, and similar terms as used herein, refer to a nucleic acid sequence that is transcribed (in the case of DNA) and translated (in the case of mRNA) into a polypeptide in vitro or in vivo when placed under control of appropriate regulatory sequences.

### DETAILED DESCRIPTION

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

As embodied and broadly described herein, the present invention is directed to the surprising finding that a population of stem cells derived, for example induced pluripotent stem cell derived, cardiomyocytes with both a matured electrophysiological phenotype and optical excitability for high-fidelity beating frequency modulation may be obtained. With the method of the invention, stem cell derived cardiomyocytes, e.g. a population of stem cell derived cardiomyocytes, may obtained that show a mature phenotype in comparison to the same stem cell derived cardiomyocytes not subjected to the method of the invention disclosed herein, the latter displaying an immature phenotype. In other words, the method of the invention provides for matured stem cell derived cardiomyocytes obtained from stem cell derived cardiomyocytes with an immature phenotype.

More in particular, it was found that it is possible to provide for such population of stem cell derived cardiomyocytes with a matured electrophysiological phenotype and with an optical excitability for high-fidelity beating frequency modulation (pacing) by introducing, in accordance with the methods of the invention, a nucleic acid encoding a light-sensitive ion channel and by introducing a nucleic acid encoding a Kir2.x channel in such population of stem cell derived cardiomyocytes (either to the cardiomyocytes or to the stem cells from which the cardiomyocytes are differentiated in vitro).

It will be understood by the skilled person that the nucleic acids as taught herein may be introduced in the cells before stem cells are differentiated into a cardiomyocytes, during the period that stem cells are differentiated into cardiomyocytes and/or after the stem cells have been differentiated into cardiomyocytes, using method well-known to the skilled person. It will also be understood that, for example, either a nucleic acid encoding the light-sensitive ion channel or a nucleic acid encoding the Kir2.x channel is introduced in stem cells before or during differentiation (in other words, one of the two nucleic acids is introduced first) and that the other is introduced (in the same cell) at another stage of differentiation of the stem cell, for example, after the stem cell differentiated into cardiomyocytes. For instance, it can be that the nucleic acid encoding the light-sensitive ion channel is introduced in the stem cells before or during differentiation and that the nucleic acid encoding the Kir2.x channel is introduced at another stage of differentiation of the same stem cell, for example, after the stem cell differentiated into cardiomyocytes, and vice versa. This ultimately would lead to cardiomyocytes having the nucleic acid encoding the light-sensitive ion channel and/or the nucleic acid encoding the Kir2.x channel.

In other words, everywhere herein were reference is made to “Introducing a nucleic acid encoding a light-sensitive ion channel and/or the Kir2.x channel in the stem cell derived cardiomyocytes”, i.e. in a population of such cells, or the like, it is also to be understood that this encompasses introducing of one, or both (although they can be introduced separately, one at a time, over time), in the same stem cells to be differentiated into the cardiomyocytes.

Likewise, it will be understood by the skilled person that everywhere herein where reference is made to “Providing stem cell derived cardiomyocytes”, this may, in certain embodiments, indicate that stem cells to be differentiated into cardiomyocytes are provided.

However, in order to keep the description concise these intended embodiments and options will not be repeated in words in any and every instance herein, although these are clearly encompassed as part of the current invention.

The (population of) stem cell derived cardiomyocytes expressing the light-sensitive ion channel and the Kir2.x channel show an advantageous phenotype that makes these cell in particular suitable for use in screening methods, for example, in screening a drug candidate for cardiotoxicity, screening in cardiomyocyte disease models for drug efficacy and for use in methods of optically inducing cardiomyocyte contraction. Importantly, and surprisingly, the population of stem cell derived cardiomyocytes expressing a light-sensitive ion channel and a Kir2.x channel can be prevented from dedifferentiation and be promoted to further mature. This beneficial characteristic cannot be observed in a population of control stem cell derived cardiomyocytes wherein no nucleic acid has been introduced or wherein only one of the nucleic acids encoding the light sensitive ion channel or encoding the Kir2.x channel is introduced.

Therefore, by the introduction, in accordance with the invention, of a light-sensitive ion channel and a Kir2.x-channel in a population of stem cell derived cardiomyocytes, for the first time, and surprisingly, a population of stem cell derived cardiomyocytes displaying the above advantageous phenotypical features are obtained. These cardiomyocytes can now be used for standardized assays under precise control of beat rate to precisely identify efficacious and toxic compounds by pacing at various pacing frequencies for example in an MEA assay.

Thus, according to the invention there is provided for an in vitro method for providing stem cell derived cardiomyocytes, the method comprising

a) Providing stem cell derived cardiomyocytes (or stem cells);

b) Introducing a nucleic acid encoding a light-sensitive ion channel in said stem cell derived cardiomyocytes (or stem cells);

c) Introducing a nucleic acid encoding a Kir2.xl channel in said stem cell derived cardiomyocytes (or stem cells); and

d) Allowing the stem cell derived cardiomyocytes to express the light-sensitive ion channel and the Kir2.x channel (where, in the case a nucleic acid is introduced in stem cells, said stem cells are differentiated in vitro into cardiomyocytes, as taught above).

In other words, according to the invention, in a step there is provided for (a population of) stem cell derived cardiomyocytes. The cells may be provided, for example, as part of a culture of stem cell derived cardiomyocytes, for example in the form of a monolayer on a cultivation dish. The stem cell derived cardiomyocytes are preferably stem cell derived cardiomyocytes obtained by in vitro differentiation of stem cells.

As explained herein, it will be understood by the skilled person that the order of events in any method or use claim may not necessarily be in the order it is presented. For example, within the context of the current invention, the order of events a), b), and c) in the in vitro method for providing stem cell derived cardiomyocytes according to the invention may be in any order suitable, for example a), b), c), or b), c), a), or b), a), c). For example, in the second example, the nucleic acid encoding the light-sensitive ion channel and the nucleic acid encoding the Kir2.x may be introduced in the stem cell before it is differentiated in vitro to cardiomyocytes. In that case the method would thus comprise

b) Introducing a nucleic acid encoding a light-sensitive ion channel in stem cells;

c) Introducing a nucleic acid encoding a Kir2.x channel in the stem cells; and

a) Providing stem cell derived cardiomyocytes (from the stem cells wherein the light-sensitive ion channel and the Kir2.x channel have been introduced, by in vitro differentiating the stem cells into cardiomyocytes);

It will also be understood by the skilled person that not each and any cell in the population of stem cells/stem cell derived cardiomyocytes necessarily needs to express both the Kir2.x channel and the light-sensitive ion channel. Indeed, only a subpopulation of cells may express, for example, at least the Kir2.x channel, and another subpopulation may, at least express the light-sensitive ion channel, whereas another subpopulation expresses both or none. It will thus be appreciated that in one embodiment, the steps b) and c) above may be performed on two independent populations of cells or on the same population of cells. In the first case, the two populations of cells may be combined to form the population of cells that in step d) are allowed to express the light-sensitive ion channel and the Kir2.x channel.

In some embodiments, Kir2.x expression in the cells is not induced or increased by the exogenous addition of a nucleic acid encoding the same, but, for example by modifying expression of the internal Kir2.x channel by genetic means./nlp

The skilled person knows how to provide for such (population of) stem cell derived cardiomyocytes, and by using methods known in the art, for example as described herein or referred herein elsewhere. As stated, the stem cell derived cardiomyocytes are preferably stem cell derived cardiomyocytes that are obtained by in vitro cultivation of stem cells under appropriate conditions. Non-limiting examples of cultivation methods are described in EP3008172, EP3152300, WO2016133392, WO2017039445 and the like.

The stem cell derived cardiomyocytes may be immature stem cell derived cardiomyocytes (also referred to as a fetal-like cardiomyocytes), for example characterized by spontaneous contraction of the stem cell derived cardiomyocytes, for example as can be witnessed by observing a monolayer of such in vitro cultivated stem cell derived cardiomyocytes. Or they are a mixed population each of which may be considered more or less mature but due to the presence of Atrial like cells and Nodal like cells still spontaneously beat.

The stem cell derived cardiomyocytes (or stem cells) are treated with the goal of introducing a nucleic acid in the stem cell derived cardiomyocytes. Such methods of introducing nucleic acids in the cells are well-known to the skilled person. The nucleic acid employed in the method of the current invention may a heterologous nucleic acids or a homologous nucleic acid, i.e. may be a nucleic acid that originates from a different or from the same organisms as the stem cell derived cardiomyocytes wherein the nucleic acid is to be introduced. In other words, the nucleic acid may be a nucleic acid that does or does not naturally exist in the stem cell derived cardiomyocytes.

Typically a nucleic acid may be introduced as a vector construct incorporating the nucleic acid sequence and subsequently introduced into the host cell via a number of methods known in the art. Standard techniques for the construction of expression vectors suitable for use in the present invention are well-known to one of ordinary skill in the art and can be found in such publications such as Sambrook J, et al., “Molecular cloning: a laboratory manual,” (3rd ed. Cold Spring Harbor Press, Cold Spring Harbor, N.Y. 2001).

Non-limiting examples of techniques suitable for introducing a nucleic acid in stem cell derived cardiomyocytes include calcium phosphate precipitation, treatment with liposomes containing the nucleic acid, DEAE dextran, electroporation and micro-injection of the DNA or RNA directly into the cells. Alternatively, the nucleic acid is introduced in the stem cell derived cardiomyocytes using viral-mediated nucleic acid delivery. Viral-mediated nucleic acid delivery is well-known to the skilled person and includes for example, the use of recombinant viruses including retrovirus, adenovirus, herpesvirus, pox virus, and adeno-associated virus (AAV). As stated, DNA and or viral vectors may be introduced prior to differentiation as stable integrations into the chromosomes or as extra-chromosomal DNA (artificial chromosome, BacMam etc).

Therefore, according some embodiments of the invention the nucleic acid may be provided to the cells in the form of an isolated nucleic acid, e.g., a plasmid-based vector which may be inserted into the genome or extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes.

In some embodiments, non-viral mediated techniques for introducing the nucleic acid includes, for example, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like), micelle fusion or cell fusion. Other methods of transfection include proprietary transfection reagents such as Lipofectamine™, Dojindo Hilymax™, Fugene™, jetPEI™, Effectene™ and DreamFect™ Fuselt™ or Xpress.4U™.

Expression of the nucleic acid is normally achieved by operably linking the encoding DNA or cDNA to a promoter (which is either constitutive, tissue specific, up or down regulated or inducible), followed by incorporation into an expression vector, for example a plasmid. The vectors can be suitable for replication and/or integration in the stem cell derived cardiomyocytes. Typical expression vectors contain transcription and translation terminators, initiation sequences, and promoters useful for regulation of the expression of the DNA encoding the enzymes. To obtain high level expression, it may be desirable to construct expression plasmids which contain a strong promoter to direct transcription, tissue specific promoter, developmental stage specific promoter a ribosome binding site for translational initiation, and IRES site for concatenating separate reading frames and a transcription/translation terminator.

The introduced nucleic acids may be expressed stable or transient. In a preferred embodiment, the introduced nucleic acids are expressed transiently, for example for a period of time of between about 12 hours and 6 weeks, for example between 12 hours and at least one week, for example, between 12 hours and 4 days, 3 days or 2 days.

In step b) the nucleic acid introduced encodes a light-sensitive ion channel.

Light-gated ion channels or light-sensitive ion channels are known to the skilled person and are transmembrane proteins that form an ion channel that opens and/or closes in response to light. The kind of light-sensitive ion channel for use in the current invention is not limited in particular as long as the light-sensitive ion channel forms an ion channel that opens and/or closes in response to light, i.e. as long as the light-sensitive ion channel formed is functional in the stem cell derived cardiomyocytes. Examples of such light-sensitive ion channels are described herein, and have recently been reviewed by Deisseroth et al. (Science 2017; 357(6356). pii: eaan5544. doi: 10.1126/science.aan 5544).

In step c) the nucleic acid introduced encodes a Kir2.x channel.

Wherein herein reference is made to a Kir2.x channel, it is to be understood that reference is made to a Kir2.1 channel, a Kir2.2 channel, a Kir2.3 channel and/or a Kir2.4 channel (and/or nucleic acid encoding such). Preferably the channel is Kir2.1.

In other words, the potassium (K) ion channel Kir2.x is selected from the group consisting of Kir2.1, Kir2.2, Kir2.3, and Kir2.4

Although there are no particular limitations on the inwardly rectifying K ion channel, preferred examples include various types of Kir2.x channels such as Kir2.1, 2.2, 2.3 and 2.4 channels. The Kir2.1 channel is an inwardly rectifying K+ channel having a two-pass transmembrane structure. This channel is not dependent on voltage and has the property to make membrane potential approaching the K+ equilibrium potential. This channel is expressed in nerves, heart and skeletal muscle, and carries out formation of resting membrane potential along with its stabilization and maintenance. Another example is Kir2.2. Although Kir2.2 is also an inwardly rectifying K ion channel in the same manner as Kir2.1, it has more potent inward rectification than Kir2.1. It is expressed with Kir2.1 in heart, brain and skeletal muscle, and plays a leading role among other inwardly rectifying K ion channels in human vascular endothelial cells.

The Kir2.x ion channels are described in the art, for example in Circ. Res. 2004, 94, 1332-1339 and Am. J. Physiol. Cell Physiol. 2005, 289, C1134-C1144. Examples of base sequences encoding human-derived Kir 2.x channels include Kir 2.1 (GenBank Accession No. U12507, NM-00891.2), Kir 2.2 (GenBank Accession No. AB074970, NM-021012 (Human KCNJ12)), Kir 2.3 (GenBank Accession No. U07364, U24056) and Kir 2.4 (GenBank Accession No. AF081466.1).

As stated Kir2.x channels, including Kir2.1 channels are known in the art and are multi-subunit channels, for which four monomeric subunits co-assemble following expression within a cell. As used herein, the terms “KCNJ2” and “Kir2.1” both refer to the Potassium Channel, Inwardly Rectifying Subfamily J, Member 2, which may also be referred to in the literature by a number of other aliases including Potassium Inwardly-Rectifying Channel, Subfamily J, Member 2, Cardiac Inward Rectifier Potassium Channel, IRK1, IRK-1, HIRK1, HHIRK1, and ATFB9.

Potassium channels are present in most mammalian cells, where they participate in a wide range of physiologic responses. The protein encoded by this gene is an integral membrane protein and inward-rectifier type potassium channel. Kir2.1 is believed to play a role in establishing action potential waveform and excitability of neuronal and muscle tissues. RefSeq DNA sequences for KCNJ2 include the following: NC_000017.11, NT_010783.16, and NC_018928.2. The KCNJ2 gene encodes a protein of 427 amino acids having a mass of 48288 Da (48.3 kDa). The Protein Symbol is P63252-KCNJ2_HUMAN, its recommended name is Inward rectifier potassium channel 2; its Protein Accession No. is P63252; and secondary accession numbers include 015110 and P48049. An extensive review on Kir2.1 channels and function was published by Willis et al. (Am J Physiol Heart Circ Physiol. 2015; 308(12):H1463-73. doi: 10.1152/ajpheart.00176.2015). Also contemplated are mutated Kir2.x channels, such as a mutated Kir2.1 channel, Kir2.2 channel, Kir2.3 channel and/or Kir2.4 channel or modified Kir2.x channels such as a modified Kir2.1 channel, Kir2.2 channel, Kir2.3 channel and/or a Kir2.4 channel that have maintained their function, i.e. that still acts as a potassium channel like the corresponding Kir2.1 channel, Kir2.2 channel, Kir2.3 channel and/or a Kir2.4 channel.

After introduction of the nucleic acids in step b) and step c), the (population of) stem cell derived cardiomyocytes are allowed to express the light-sensitive ion channel and the Kir2.x channel. In case nucleic acids have been introduced in the stem cells or during differentiation of the stem cells, the cells are allowed to further differentiate towards cardiomyocytes, as will be understood by the skilled person.

The step of allowing the expression of the light-sensitive ion channel and the Kir2.x channel is not limited by any particular procedure and may be provided for by any technique well-known to the skilled person.

Although the type of nucleic acid encoding a light sensitive ion channel and introduced in the stem cell derived cardiomyocytes is not limited in particular, and may, for example comprise DNA, RNA, cDNA, genomic DNA, or a hybrid of the various combinations, in an embodiment the nucleic acid is RNA, more in particular messenger RNA, mRNA, encoding a (functional) light-sensitive ion channel protein.

Although the type of nucleic acid encoding the Kir2.x channel and introduced in the cells is not limited in particular, and may, for example comprise DNA, RNA, cDNA, genomic DNA, or a hybrid of the various combinations, in an embodiment the nucleic acid is RNA, more in particular messenger RNA, mRNA, encoding a Kir2.x channel.

Within the context of the current invention, the term mRNA refers to a RNA molecule that can be translated into a polypeptide. Such mRNA may be synthetically prepared or be obtained by transcription of the corresponding DNA molecule and subsequently purified.

The mRNA may be modified or be unmodified. The mRNA may comprise analog nucleotides not naturally occurring or not. For example, the mRNA may be a capped RNA, i.e. a RNA molecule comprising at least one cap nucleotide. A cap or a cap nucleotide means a nucleoside-5′-triphosphate that, under suitable reaction conditions, is used as a substrate by a capping enzyme system and that is thereby joined to the 5′-end of an uncapped RNA, for example comprising primary RNA transcripts or RNA having a 5′-diphosphate. The nucleotide that is so joined to the RNA is also referred to as a cap nucleotide. A cap nucleotide is normally a guanine nucleotide that is joined through its 5′ end to the 5′ end of a (primary) RNA transcript. The RNA that has the cap nucleotide joined to its 5′ end is referred to as capped RNA. A common cap nucleoside is 7-methylguanosine or N7-methylguanosine (sometimes referred to as “standard cap”). For example, in the example provide herein, for stabilization, both mRNAs carry a m7G and a methylated 2′-hydroxy group on the first ribose sugar at the 5′end. This structure is added by the mRNA vendor during production and is called CleanCap (trilinkbiotech.com/cleancap/).

Therefore, in such embodiments there is provided for the in vitro method as described herein wherein the nucleic acid encoding a (functional) light-sensitive ion channel is a mRNA encoding a (functional) light-sensitive ion channel and/or wherein the nucleic acid encoding a (functional) Kir2.x channel is a mRNA encoding a (functional) Kir2.x channel. Preferably both the light-sensitive ion channel and the Kir2.x channel are introduced using as a nucleic acid a mRNA encoding for the light-sensitive ion channel and a mRNA for the Kir2.x channel. In a specific embodiment the two functional proteins may be fused into one continuous protein or joined using a linker or transfected as one construct where two ORF are separated by a IRES site (or similar).

Functional in the case of a Kir2.x channel or gene refers to a lowering of resting membrane potential. In the case of light activated ion channels “functional” refers to the ability to open the ion channel allowing the passage of ions more freely eliciting an action potential in response to a light stimulus. Functional proteins may be attenuated, truncated, fused, or contain deletions as well as insertions.

The mRNA may be introduced by methods well-known to the skilled person, including such methods as described in the Example. In some embodiments, the step of introducing mRNA comprises delivering the mRNA into the cell with a transfection reagent. However, the invention is not limited by the nature of the transfection method utilized. Indeed, any transfection process known, or identified in the future that is able to deliver mRNA molecules into cells in vitro, is contemplated, including methods that deliver the mRNA into cells in culture. In some embodiments, the transfection reagent comprises a lipid (e.g., liposomes, micelles, etc.). In some embodiments, the transfection reagent comprises a nanoparticle or nanotube. In some embodiments, the transfection reagent comprises a cationic compound (e.g., polyethylene imine or PEI). In some embodiments, the transfection method uses an electric current to deliver the mRNA into the cell (e.g., by electroporation). In some embodiments, the system is based on fusogenic liposomes (e.g. beniag GmbH) transferring mRNA molecules directly upon contact into the cytoplasm of cells.

It was found that when using mRNA for introducing the light-sensitive ion channel and/or using mRNA for introducing the Kir2.x channel in the stem cell derived cardiomyocytes, cells were obtained that display advantageous characteristics. For example, stem cell derived cardiomyocytes were obtained that do not or only to a limited degree display spontaneous contraction (a feature of immature stem cell derived cardiomyocytes) but at the same time that do not dedifferentiate (a feature often seen when cardiomyocytes lose their ability of spontaneous contraction). In addition, although expression of the light-sensitive ion channel and of the Kir2.x channel is transient, it was found that when using mRNA for introducing expression of the light-sensitive ion channel and using mRNA for introducing the expression of the Kir2.x channel, expression was sufficiently long and adequate to allow for use of these cells in, for example, methods of screening for cardiotoxicity.

As discussed above, not all cells necessarily need to be transfected by both factors (ion channel and Kir2.x) but one population (the main population) may be transfected by Kir2.x lowering the resting membrane potential and a second population or a subset of the first population may be transfected with the light sensitive ion channel. This second population even though it may be at lower number (e.g. between 100% and 90%, 80%, 70%, 60%, 50%, 40%, 30%, 20%, 10%, 5% as compared to the first population), and not in all cells, can trigger the action potential in the whole synthitium. The populations may for example be co-cultured in a continuous 2D (monolayer) or 3 dimensional (3D) culture.

Thus be employing the methods according to the invention it is now possible to normally cultivate and obtain stem cell derived cardiomyocytes, without the need of the exogenous introduction of any nucleic acid and which introduction at an early stage of cultivation may severely influence the fate of the thus cultivated stem cell derived cell. For example, only shortly before the cells are to be used, for example in a screening assay, steps b) and c) are performed, thereby introducing a nucleic acid encoding a light-sensitive ion channel and introducing a nucleic acid encoding a Kir2.x channel in the stem cell derived stem cells. Thus in one embodiment steps b) and/or step c) of the method of the invention is/are performed no more than 96 hours, preferably no more than 72, 60, 48 hours before the cells are employed in, for example, a screening assay, for example as detailed herein.

In some embodiments there is provided for the in vitro method disclosed herein wherein a subpopulation of the stem cell derived cardiomyocytes provided in step a) are treated according to step b) and wherein another subpopulation of the stem cell derived cardiomyocytes provided in step a) are treated according to step c), and wherein the subpopulations are combined after the treatments of step b) and c).

As explained herein, it is not required that step b) and step c) are performed simultaneously or on the same population of cells. It is also envisaged that a part of the stem cell derived cardiomyocytes of step a) are treated according to step b) and a part of the stem cell derived cardiomyocytes is treated according to step c). After performing the steps b) and c) the subpopulations may be combined and co-cultured to provide for a population of stem cell derived cardiomyocytes wherein a part expresses the Kir2.x and another part expresses the light sensitive ion channel protein. Thus, also provided is the in vitro method wherein the stem cell derived cardiomyocytes of step d) comprises stem cell derived cardiomyocytes that express Kir2.x and comprises stem cell derived cardiomyocytes that express the light-sensitive ion channel.

As explained, the order in which steps a), b) and c) are performed are not limited in particular. This transfection as in b and/or c may be done before the differentiation step leading to an in vitro differentiated cardiomyocytes. Thus, in some embodiments step b) is performed before step c) is performed. In some embodiments step c) is performed before step b). In some embodiments step b) and c) are performed at the same time or concurrently. Performing step b) and step c) may overlap in part or may be separate.

Thus, for example, first step b) may be performed, and after step b) has been performed step c) is performed. Alternatively, first step b) is performed, and while step b) is being performed, step c) is performed.

However, in a preferred embodiment step b) is performed before step c) is performed (and wherein step b) and step c) are performed separate from each other or overlap for at least some time in the same of in two parts of the population of cells. It was surprisingly found that when first step b) is performed, and followed in time by step c), (a populations) of stem cell derived cardiomyocytes may be obtained with improved characteristics, for example with respect to the expression of the light-sensitive ion channel and the Kir2.x channel. The cells thus obtained were found to be in particular useful, for example in the screening methods and/or methods for optically inducing cardiomyocyte contraction and/or for preventing dedifferentiation and/or for promoting maturation, for example as disclosed herein.

In other words, it was found that in a preferred embodiment, the stem cell derived cardiomyocytes are first contacted with a nucleic acid encoding a light-sensitive ion channel, i.e. a functional light-sensitive channel, and then contacted with a nucleic acid encoding a Kir2.x channel, i.e. a functional Kir2.x channel.

Light-gated ion channels or light-sensitive ion channels are transmembrane proteins that form an ion channel that opens and/or closes in response to light. The kind of light-sensitive ion channel for use in the current invention is not limited in particular as long as the light-sensitive ion channel forms an ion channel that opens and/or closes in response to light.

Light-sensitive ion channels useful in the present invention include, but are not limited to those mentioned herein, as well as mutations and other variations and alterations of the light-sensitive ion channels such as chimeras and light-sensitive ion channels comprising one or more deletions or insertions. This includes variants found in nature as well as novel mutations and codon usage optimizations. These may include wholly synthetic de novo developments of amino acid sequences which exhibit a comparable effect. Such mutations or alterations may, for example, alter the wavelength of light at which the light-sensitive channel is activated, alter the specificity of the channel for certain ions, and/or alter the kinetics of the light-gated ion channel.

One well-knows class of light-sensitive ion channels are the channelrhodopsins. Channelrhodopsins are a subfamily of retinylidene proteins (rhodopsins), comprising, for example Channelrhodopsin 1 and Channelrhodopsin 2 (ChR2). For example, the wild-type ChR2 absorbs blue light with an absorption and action spectrum maximum at 480 nm. C128 or D156 mutations of Channelrhodopsin 2 (ChR2) provide a light-sensitive ion channel that can be opened by a blue light pulse and closed by a green or yellow light pulse. Mutations of the E123 residue may accelerate channel kinetics (ChETA). A C-terminal truncation of ChR2 (ChR21-315) is reported to be substantially as active as the full-length protein. Light-sensitive ion channels also include variations that have been mammalian codon-optimized. Also possible are chimera of a light-sensitive ion channel comprising parts of more than one type of light-sensitive (gated) ion channels. Chimeric channelrhodopsins have been developed by combining transmembrane helices from ChR1 and VChR1, leading to the development of ChRs with red spectral shifts (such as C1V1 and ReaChR).

Thus, in one embodiment, the light-sensitive ion channel protein is one or more proteins selected from the group consisting of channelrhodopsin-1 (ChR1), channelrhodopsin-2(ChR2), Volvox channelrhodopsin (VChR1), halorhodopsin (Halo/NpHR), archaerhodopsin-3 (Arch), and Leptosphaeria maculans rhodopsin (Mac).

According to a preferred embodiment, the light-sensitive ion channel protein is ChR1 or ChR2, chimera comprising parts of ChR1 and/or ChR2 or mutated versions thereof that are functional, i.e. that opens and/or closes in response to light.

The kind of stem cell from which the cardiomyocytes are derived is not limited in particular, as long as the cells have the nature of stem cells, that is, abilities to divide in culture and to differentiate into one or more cell types, here, in particular into cardiomyocytes. Such stem cells may, for example by embryonic stem cells, pluripotent stem cells, induced pluripotent stem cells, embryonic germ cells, embryonal carcinoma cells (EC cell), multipotent adult progenitor cells (MAP cell), or adult pluripotent stem cells (APS cell). The stem cells may be embryonic or somatic stem cells. For example, the above-mentioned pluripotent stem cells may be derived from a fetus, or may be adult tissue-derived stem cells.

From the viewpoint of cell source and ethics, the stem cell is preferred to be a cell except an embryonic stem cell, more preferred to be an induced pluripotent stem cell. In accordance therewith, in some embodiments, the stem cell derived cardiomyocytes provided in step a) are a pluripotent stem cell derived cardiomyocytes or are induced pluripotent stem cell derived cardiomyocytes.

In a preferred embodiment, the stem cell derived cardiomyocytes provided in step a) of the method described above are stem cells derived cardiomyocytes that display a fetal phenotype. In a preferred embodiment, the stem cell derived cardiomyocytes provide is step a) of the method described above, are stem cells that exhibits automaticity (spontaneous) contraction. Such stem cell derived cardiomyocytes may also exhibit fetal-type ion channel expression, and/or fetal-type electrophysiological signals.

Therefore, in some embodiments, the stem cell derived cardiomyocytes provided in step a) are (differentiated to) spontaneous beating stem cell derived cardiomyocytes. The skilled person is well aware of such spontaneous beating stem cell derived cardiomyocytes and how to obtain these or how to identify these, for example as described in the Example section herein.

The species of the stem cell from which the cardiomyocytes are derived is not limited in particular, however it was found that the current invention is in particular suitable for stem cell derived cardiomyocytes from mammalian origin, in particular from human origin. Therefore, in some embodiments, the stem cell derived cardiomyocytes provided in step a) of the method described above are mammalian stem cell derived cardiomyocytes, preferably human stem cell derived cardiomyocytes.

As disclosed herein, the kind of light-sensitive ion channel used in the method of the invention is not limited in particular, as long as the ion channel can be expressed functionally in the stem cell derived cardiomyocytes, i.e. it forms an ion channel that opens and/or closes in response to light.

In some embodiments, the nucleic acid encoding a light-sensitive ion channel encodes a fusion protein comprising the light-sensitive ion channel. The functional light-sensitive ion channel may, for example, be fused to a reporter protein allowing to monitor expression of the fusion protein in the stem cell derived cardiomyocytes after introduction of the nucleic acid. It may, for example also be fused to an amino acid sequence for improved routing of the light-sensitive ion channel to the desired cellular compartment.

In some embodiments of the invention, for example of the in vitro method disclosed herein, the nucleic acid encoding a light-sensitive ion channel is a nucleic acid selected from

a) a nucleic acid having a sequence as shown in SEQ ID NO: 1.

b) a nucleic acid having a sequence that is transcribed in a sequence as shown in SEQ ID NO: 1; or

c) a nucleic acid encoding a polypeptide having at least 80% sequence identity with a polypeptide encoded by the nucleic acid of a) or b) above.

The nucleic acid sequence of SEQ ID No:1 is a RNA sequence. The nucleic acid having a sequence that is transcribed in a sequence as shown in SEQ ID NO: 1 may, for example be a DNA molecule comprising a coding region that when transcribed into mRNA provides for a sequence as is shown in SEQ ID No:1. A nucleotide encoding a polypeptide having at least 80%, 85%, 90%, 85%, 98%, 99% sequence identity with a polypeptide encoded by the nucleic acid of a) or b) above may be a DNA molecule comprising a coding region that, when transcribed into mRNA and translated into a polypeptide provides a polypeptide that has at least 80%, 85%, 90%, 85%, 98%, 99% sequence identity with a polypeptide encoded by the nucleic acid of a) or b). The polypeptide is a functional polypeptide.

Likewise provided is that, in some embodiments, the nucleic acid encoding a Kir2.x, in particular a Kir2.1 channel is a nucleic acid selected from

a) a nucleic acid having a sequence as shown in SEQ ID NO: 2.

b) a nucleic acid having a sequence that is transcribed in a sequence as shown in SEQ ID NO: 2; or

c) a nucleic acid encoding a polypeptide having at least 80% sequence identity with a polypeptide encoded by the nucleic acid of a) or b) above.

The nucleic acid sequence of SEQ ID No:2 is a RNA sequence. The nucleic acid having a sequence that is transcribed in a sequence as shown in SEQ ID NO: 2 may, for example be a DNA molecule comprising a coding region that when transcribed into mRNA provides for a sequence as is shown in SEQ ID No:1. A nucleotide encoding a polypeptide having at least 80%, 85%, 90%, 85%, 98%, 99% sequence identity with a polypeptide encoded by the nucleic acid of a) or b) above may be a DNA molecule comprising a coding region that, when transcribed into mRNA and translated into a polypeptide provides a polypeptide that has at least 80%, 85%, 90%, 85%, 98%, 99% sequence identity with a polypeptide encoded by the nucleic acid of a) or b). The polypeptide is a functional polypeptide.

Two polynucleotides or polypeptides are said to be identical if the sequence of nucleotides or amino acid residues in the two sequences is the same when aligned for maximum correspondence. Optimal alignment of sequences for comparison may be conducted by the local homology algorithm of Smith and Waterman Adv. Appl. Math. 2: 482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. Substantial identity means that a polypeptide comprises a sequence that has at least 80% sequence identity, preferably 90%, more preferably 95% or more, compared to a reference sequence over a comparison window of about 20 residues to about 600 residues—typically about 50, 60, 100 to about 200, 300, 400 Or 500. The values of percent identity are determined using the programs above.

It was found that when the nucleic acid that is introduced in the stem cell derived cardiomyocytes is a mRNA encoding a light-sensitive ion channel and/or wherein the nucleic acid is a mRNA encoding a Kir2.x channel, the amount of mRNA is not limited in particular, but is preferably about 0.01-1.5 microgram mRNA per 10.000 provided stem cell derived cardiomyocytes, preferably about 0.1-0.9 microgram mRNA per 10.000 provided stem cell derived cardiomyocytes.

It was found that such preferred amounts of mRNA provide for stem cells according to the invention that are optimal for use in methods for screening for cardiotoxicity of candidate drugs or compounds, for optically inducing cardiomyocyte contraction, for use in methods of preventing dedifferentiation and for use in methods for promoting maturation of the stem cell derived cardiomyocytes.

However, the skilled person understands that also more or less mRNA per 10.000 provided stem cells may be used in step b) or step c) of the method described above.

The time duration of step b) and/or step c) as described above is not limited in particular. For example, step b) may be performed from between 5 minutes and 21 days. For example, step c) may be performed from between 5 minutes and 21 days. In other words, the stem cell derived cardiomyocytes may be contacted with the nucleic acid in step b) or step a) for such period, for example by providing the nucleic acid to the medium in which the cells are cultivated or maintained.

As stated herein, transfection of light sensitive ion channel may be done only to a subset of cells or in a different population of cells which are later co-cultured with the Kir 2.x transfected cells.

However, in a preferred embodiment of the in vitro method as disclosed herein, step b) is performed between 5 minutes and 24 hours and step c) is performed between 5 minutes and 24 hours or wherein step b) and c) is performed between 5 minutes and 24 hours.

As described herein, preferably step b) is performed before step c) is performed. Preferably step c) is performed 6-48, preferably 12-24 hours after the start of step b). Preferably, the nucleic acid used in step b) and/or step c) is a RNA molecule, in particular mRNA.

It was found that, in case use is made of mRNA, the light-sensitive ion channel, e.g. ChR2, may preferably be transfected first, although this is not necessary. It was surprisingly observed that the peak of expression of the light sensitive ion channel is reached later post-transfection that the peak of expression of the Kir2.x channel. In fact, by first introducing the light-sensitive ion channel and then introducing the Kir2.x channel, optimal expression of both at the same time may be obtained. In addition, since expression is transient, the cells become and remain quiescent after introduction of the Kir2.x channel for a limited period of time.

The in vitro method as described herein may be performed using different types of culturing systems. However, in a preferred embodiment the in vitro method is performed using a multi-electrode array plate, preferably a multi-well multi-electrode plate. In other words, the stem cell derived cardiomyocytes that are provided in step a) of the method of the invention are plated on a multi-electrode array system (e.g. MEA, Multichannel Systems, Reutlingen, Germany).

In another embodiment, the stem cell derived cardiomyocytes of step a) are provided as a monolayer of stem cell-derived cardiomyocytes, in particular stem-cell derived cardiomyocytes that exhibit spontaneous or automaticity (spontaneous) contraction. It was found that when the stem cell derived cardiomyocytes of step a) are provided as a monolayer of stem cell derived cardiomyocytes, performing the other steps of the method disclosed herein provides for stem cell derived cardiomyocytes that can advantageously be used in, for example, screening compound for cardiotoxicity, in methods of optically inducing cardiomyocyte contraction and/or methods of preventing dedifferentiation of the stem cell derived cardiomyocytes and/or methods of promoting maturation of the stem cell derived cardiomyocytes.

It was also found that heterogeneous populations can, with the method of the invention, be stimulated to become more homogeneous (spontaneous beating, atrial, ventricular nodal like genotype as seen in spontaneous action potentials.

According to an aspect of the invention there is provided for (a population of) stem cell derived cardiomyocytes that are obtainable with the method of any of the previous claims. Such stem cell derived cardiomyocytes may display advantageous characteristics including those described herein in detail.

Also provided is for a population of or stem cell derived cardiomyocytes wherein the stem cell derived cardiomyocytes (or the population) comprises a nucleic acid encoding a light-sensitive ion channel and comprises a nucleic acid encoding a Kir2.x channel. Preferably the nucleic acid is a RNA molecule, preferably an mRNA molecule. Preferably, the nucleic acid is non-integrated in the chromosome of the stem cell derived cardiomyocytes.

According to a preferred embodiment, there is provided for (a population of) stem cell derived cardiomyocytes, preferably wherein the (population of) stem cell derived cardiomyocytes comprises a nucleic acid encoding a light-sensitive ion channel and comprises a nucleic acid encoding a Kir2.x channel, wherein the (population of) stem cell derived cardiomyocytes expresses the light-sensitive ion channel, preferably by the exogenous introduction of mRNA encoding the light-sensitive ion channel and expresses a Kir2.x channel, preferably by the introduction of mRNA encoding the Kir2.x channel.

In a preferred embodiment, the stem cell derived cardiomyocytes of the invention are (a population of) stem cell derived cardiomyocytes wherein the light-sensitive ion channel and the Kir2.x are overexpressed relative to unmodified stem cell derived cardiomyocytes. The skilled person is well-known with methods for comparing level of expression of the light-sensitive ion channel and/or the Kir2.x. Overexpression may, for example be defined, as any protein expression that is at least 10%, 20%, 50%, 100%, 200%, 500% more than the expression measured in unmodified stem cell derived cardiomyocytes, i.e. the stem cell derived cardiomyocytes in which no nucleic acid encoding for the light-sensitive ion channel is introduced and/or in which no nucleic acid encoding for the Kir2 channel is introduced.

According to another aspect of the current invention there is provide for a combination, kit or kit-op-parts comprising

a) Stem cell derived cardiomyocytes, nucleic acid encoding a light-sensitive ion channel, and nucleic acid encoding a Kir 2.x channel; or

b) Stem cell derived cardiomyocytes or population of stem cell derived cardiomyocytes obtainable by the method of the invention (expressing a light-sensitive ion channel and/or a Kir2.x channel, as taught herein).

c) A device, preferably a culture dish or multi well plate or assay plate comprising stem cell derived cardiomyocytes of any of the previous claims.

Such combination, kit or kit-of parts may comprise additional features, such a cultivation media, buffers, cultivating dishes, such as MEAs and so on. With the combination, kit or kit-of parts there is provided for a high quality and standardized tool comprising stem cell derived cardiomyocytes having beneficial characteristics, such as those disclosed herein, and that makes these cell in particular advantageous for use in methods of screening for cardiotoxicity of candidate compounds, for use in methods of optically inducing cardiomyocyte contraction, for methods of preventing dedifferentiation of stem cell derived cardiomyocytes and for methods of promoting maturation of stem cell derived cardiomyocytes. The kits may be for use in drug screening, drug discovery and/or drug efficacy testing, for example, as described herein.

Also provided is for a culture system comprising (a population of) stem cell derived cardiomyocytes obtainable with the methods according to the invention. Such culture system may, for example a cultivating dish or a multi-electrode array plate, for example a multi-well multi-electrode plate.

According to another aspect of the invention there is provided for a method of screening a drug candidate for cardiotoxicity or efficacy (drug screening or discovery). The method of screening a drug candidate for cardiotoxicity or efficacy or drug discovery comprising

a) Providing stem cell derived cardiomyocytes obtainable with the method as described herein (expressing a light-sensitive ion channel and/or a Kir2.x channel, as taught herein);

b) contacting the stem cell derived cardiomyocytes of a) with a drug candidate;

c) optically activating the light-sensitive ion channel at one or multiple pacing frequencies thereby inducing contraction of the cardiomyocytes (or pacing the stem cell derived cardiomyocytes by optically activating the light-sensitive ion channel); and

d) measuring cardiomyocyte action potential, cardiomyocyte ion fluxes, cardiomyocyte field potential, impedance, contraction, movement, morphology, cardiomyocyte intracellular calcium level, velocity of conduction, or a combination thereof, thereby screening the drug candidate for cardiotoxicity.

Steps a, b, and c) of the above method may be in any order, e.g. be reversed.

In this embodiment, the invention provides a method of screening a drug candidate for cardiotoxicity of efficacy. The term cardiotoxicity and (cardio) efficacy are well known to the skilled person. Efficacy herein refers to drug discovery of a potential beneficial effect of the drugs candidate on the functioning of the heart, for example, a diseased heart. With the method of the invention, for example, drug screening may be performed to identify drugs that may be beneficial for the treatment of e.g. arrhythmia, heart failure, or any other condition of the hearts.

It will thus be understood by the skilled person that in some embodiments normal, healthy cells (expressing a light-sensitive ion channel and/or a Kir2.x channel, as taught herein) are used in the method of screening, including method of drug screening. However, in another embodiment, a disease model is used, for example such disease models (expressing a light-sensitive ion channel and/or a Kir2.x channel, as taught herein) as claimed and described in EP1745144 or any other known in the art, including models for LQTS (Long QT syndrome) and heart failure.

The method includes providing the stem cell derived cardiomyocytes according to the invention, i.e. the stem cell derived cardiomyocytes that are obtainable by introducing a nucleic acid encoding a light-sensitive ion channel and by introducing a nucleic acid encoding a Kir2.x channel, as detailed herein. The method also includes contacting said cells with a drug candidate and optically activating the light sensitive ion channel, thereby inducing contraction of the cardiomyocytes. In some embodiment the method includes pacing of the stem cell derived cardiomyocytes by optically activating the light-sensitive ion channel (see also, for example US 2015/0290285; Yu et al. Sci Rep. 2015; 5:16542. doi: 10.1038/srep16542; and Nussinovitch et al. Cardiovasc Res. 2014; 102(1):176-87. doi: 10.1093/cvr/cvu037.) The skilled person is well-known with such methods of optically activating the light-sensitive ion channel and/or of pacing the stem cell derived stem cells by optically activating the light-sensitive ion channel.

In one embodiment of the screening method the cells are optically activated before the candidate drug is provided; in other embodiments the candidate drug is provided before the cells are optically activated.

Optical activating the cells according to the invention may be at one or multiple pacing frequencies.

The method also comprises measuring the effect of optically activating in the presence or absence of the candidate drug, hereby screening the drug candidate for cardiotoxicity. In various aspects, measuring includes measuring cardiomyocyte action potential, cardiomyocyte ion fluxes, cardiomyocyte field potential, impedance contraction, movement, morphology, cardiomyocyte intracellular calcium level, velocity of conduction, or a combination thereof.

To determine cardiotoxicity of efficacy, the effect of the drug on the normal electrical stimulation and contraction may be analyzed. A drug candidate (or test compound) is intended to include any type of molecule, for example, a polynucleotide, a peptide, chemical compounds, such as organic molecules or small organic molecules, or the like, which may affect cardiomyocyte contraction. Drug candidates are also found among biomolecules including peptides, saccharides, fatty acids, steroids, purines, pyrimidines, derivatives, structural analogs or combinations thereof.

The screening methods of the present invention may be performed on a number of platforms.

The methods of the present invention may be performed, for example using a cell based assay using the cellular composition described herein. As such, the method is particularly suited to be performed in a high-throughput fashion, (i.e., 96 or 384-well plate analysis; mechanical or robotic processing).

According to another aspect of the invention, there is provided for a system for screening a drug candidate for cardiotoxicity or in drug discovery comprising:
a) stem cell derived cardiomyocytes obtainable with the method of the invention (expressing a light-sensitive ion channel and/or a Kir2.x channel, as taught herein);
b) an optical device to activate the light sensitive ion channel at one or multiple pacing frequencies;
c) optionally, a chemical delivery device for introducing the drug candidates to be screened;
d) a sensor; optionally the sensor is an optical sensor, voltage sensor, impedance sensor, ion sensor, or a combination thereof;
e) a processor comprising executable code to process data received from the sensor; and memory for storing data received from the sensor

In this embodiment, there is provided a system for screening a drug candidate for cardiotoxicity of efficacy (drug discovery). The system includes stem cell derived cardiomyocytes obtainable with the method of the invention (expressing a light-sensitive ion channel and/or a Kir2.x channel, as taught herein); an optical device to activate the light sensitive ion channel; a chemical delivery device for introducing a drug candidate to be screened; a sensor; a processor including executable code to process data received from the sensor; and memory for storing data received from the sensor. The sensor may, for example be an optical sensor, voltage sensor, ion sensor, an impedance sensor, or a combination thereof.

According to another aspect there is provided for a method of optically inducing cardiomyocyte contraction comprising:

a) providing stem cell derived cardiomyocytes obtainable with the method of the invention (expressing a light-sensitive ion channel and/or a Kir2.x channel, as taught herein); and

b) optically activating the light-sensitive ion channel thereby inducing contraction of the cardiomyocytes.

Methods of optically inducing cardiomyocyte contraction using light-sensitive ion channels are known to the skilled person. For example, in some embodiments the cardiomyocytes may be optically stimulated in a predetermined temporal pattern to test its robustness to arrhythmia. The pattern may comprise beating at a set of constant rates of (e.g. 0.5 Hz, 1 Hz, 2 Hz, 3 Hz, 4 Hz). Alternatively, the pattern may comprise a sudden step in beat-rate (e.g. from 1 Hz to 2 Hz, or from 2 Hz to 1 Hz). Alternatively, the pattern may comprise introduction of brief optical stimuli at predetermined times during an AP waveform, to test the robustness of the AP to ectopic beats. Alternatively, the pattern may comprise a ramp of gradually increasing stimulus rate, to determine the maximum frequency at which the cardiomyocytes can beat. In certain embodiments, optically pacing the cardiomyocytes are performed using an optical microscopy system, which system may use a digital micromirror device to control the spatial pattern of the illumination. Pacing may be done in the absence or presence of the pharmaceutical agent.

Also provided is for a method of preventing dedifferentiation of stem cell derived cardiomyocytes obtainable with the method of the invention (expressing a light-sensitive ion channel and/or a Kir2.x channel, as taught herein) comprising optically activating the light-sensitive ion channel to induce contraction of the cardiomyocytes thereby preventing dedifferentiation of stem cell derived cardiomyocytes.

Also provided is for a method of promoting maturation of stem cell derived cardiomyocytes obtainable with the method any of the previous claims (expressing a light-sensitive ion channel and/or a Kir2.x channel, as taught herein) comprising optically activating the light-sensitive ion channel to induce contraction of the cardiomyocytes.

Also provided is for a method of training the cardiomyocytes to build up contractile apparatus and capabilities to increase contractility, contractile plasticity (beating rate range)

It will be understood that all details, embodiments and preferences discussed with respect to one aspect of embodiment of the invention is likewise applicable to any other aspect or embodiment of the invention and that there is therefore not need to detail all such details, embodiments and preferences for all aspect separately.

Having now generally described the invention, the same will be more readily understood through reference to the following examples which is provided by way of illustration and is not intended to be limiting of the present invention.

### Examples

Materials and Methods

hIPSC derived cardiomyocytes (Cor.4U, Axiogenesis AG) were cultured on Geltrex (LDEV-Free Reduced Growth Factor Basement Membrane Matrix, ThermoFisher) coated T-75 flasks (Nunc EasYFlask, ThermoFisher) with cell densities of approximately 54,000 cells/cm2 and cultured in hIPSC-CM maintenance medium (Axiogenesis AG) for 4 days. Medium was changed every other day.

Electrode arrays on 96-well MEA plates (Axion BioSystems) were spot-coated with 6 μL/well of ice-cold Geltrex and were incubated for 30 minutes at 37° C. in a humidified chamber inside the incubator. Cells were detached from T-flask using Accumax (Innovative Cell Technologies, Inc.), collected by centrifugation (200×g, 5 min) and spot-seeded carefully on the center of each electrode array area at a concentration of 10,000 cells/well.

Cells were cultured at 37° C., 5% CO2 and medium was changed every day. After three days, hIPSC-CM were transfected with Channelrhodopsin2-(H134R)-eYFP mRNA (TriLink Biotechnologies) using the Xpress.4U transfection kit (Axiogenesis AG). The system is based on fusogenic liposomes (beniag GmbH) transferring mRNA molecules directly upon contact into the cytoplasm of cells. Therefore, 36 μL of ChR2-eYFP mRNA (1 μg/pL) were mixed with 36 μL of neutralizing solution A and sonicated for 5 minutes. Afterwards, 45 μL of solution B was added to the mix and sonicated for another 5 minutes to incorporate the neutralized mRNA into transfection competent fusogenic particles. Finally, PBS without calcium and magnesium (Sigma) was added to a final volume of 4.5 mL to the fusogenic particles and sonicated for 5 minutes. All sonication steps were performed in a cooled ultrasonic bath (approx. 4° C.). Medium was carefully removed from cells using an 8-channel vacuum aspirator adapter. Alternatively, use may be made of the Fuse-It-mRNA kit distributed by Ibidi (Cat. No. 60500). 50 μL of transfection complexes were added to each well using an electronic multichannel pipette yielding a final mRNA concentration of 0.4 μg/well. Cells were incubated at 37° C., 5% CO2 for 10 minutes. Subsequently, fusogenic solution was aspirated and wells were filled with 200 μL/well hIPSC-CM maintenance medium. 17 hours post-transfection (hpt) maintenance medium was changed for prewarmed Tyrode solution and optical pacing was performed on Axion-MEA platform (Axion BioSystems) using a prototype of a 96-well LED light delivery device (Nanion). After confirming response to optical stimulation, cells were transfected with KCNJ2 mRNA (mRNA encoding Kir2.1 channel) (TriLink Biotechnologies) as described for ChR2-eYFP mRNA, varying final mRNA concentrations between 0.3, 0.6 and 1.2 μg/well. For incubation times, three approaches were followed: either cells were incubated for 10 or 30 minutes with subsequent removal of transfection complexes and addition of fresh maintenance medium or cells were incubated for 30 minutes and complexes were diluted by adding 200 μL maintenance medium/well leading to a continued over-night exposure of the cells to the complexes. 17 hpt maintenance medium was changed for prewarmed Tyrode solution and electro-activity of co-transfected hIPSC-CMs was assessed by identifying wells with sustained quiescence in nonstimulated state and highest fidelity to accelerated stimulus frequencies during optical pacing on the aforementioned system. Pacing frequencies were varied between 0 Hz (no stimulation), 0.25 Hz, 0.5 Hz, 0.75 Hz, 1 Hz, 1.5 Hz and 2 Hz. Pulse duration was 10-20 ms and light intensity 75-100 mA. Field potential recordings were performed using AxIS 2.4.2 software (Axion Integrated Studio, Axion BioSystems).

Results

Co-transfected hIPSC-CM were analyzed with regards to their electro-activity on 96-well micro electrode array plates using the Axion Maestro MEA platform. Untreated cells showed spontaneous beating behavior with beating frequencies of 1 Hz.

The same was true for cells that were only transfected with ChR2-eYFP mRNA. Active wells were characterized by a high number of active electrodes 7 out of 8), confirming the formation of a functional syncytium.

Furthermore, transfection procedure did not show detrimental effect on spontaneous activity. Optical pacing of the cells was achieved by placing a LED light delivery device directly on top of the lid of the MEA plate sitting on the Axion Maestro platform. The LEDs were arranged according to the layout of a 96-well plate (i.e. 8 rows×12 columns). Prior to MEA measurements, maintenance medium was changed for warm Tyrode buffer to reduce light absorbance by pH indicator dye for improved light transmission through the wells and to reduce phototoxic effects due to flavonoid decomposition. Stimulation frequencies were gradually increased from 0.25 Hz (12.5 bpm) to 2 Hz (120 bpm), with pulse durations of 10-20 ms and a total power output of 75-100 mA. While control and “KCNJ2 only” cells remained unaffected by optical pacing, beating frequencies of ChR2-eYFP transfected cells could be modulated >1 Hz. For 1.5 Hz 7 out of 8 wells adapted to the optical stimulation frequency. For co-transfected cells, the majority of the wells treated with the lowest KCNJ2 mRNA concentration could be recovered from quiescence by optical stimulation. Wells were identified to show both characteristics, i.e. quiescence in the unstimulated state and high fidelity of beating frequency to optical stimuli. On the one hand, transfection of intermediate KCNJ2 mRNA concentrations resulted in relatively less silencing of spontaneous contractions of hIPSC-CM for short incubation times. On the other hand, 30 minute incubation with prolonged over-night exposure led to fair silencing of spontaneous activity and good addressability up to 1.5 Hz by light stimulation in 50% of the wells. Wells were identified to show total quiescence and high fidelity to all pacing rates.

The cells, methods and use of the inventions provide, in several embodiments, for example non-toxicity in preparing and handling due to the technologies used, the possibility of repeated transfections (or adding further nucleic acids encoding other proteins), quantitative transfection that may easily be regulated (titration) to influence the time window, assay window and expression amount and cells that can be paced at multiple frequencies to detect compound effects such as reverse use dependency and use dependency.

1. An in vitro method for providing stem cell derived cardiomyocytes, the method comprising
a) Providing stem cell derived cardiomyocytes;
b) Introducing a nucleic acid encoding a light-sensitive ion channel in (at least part of) the stem cell derived cardiomyocytes;
c) Introducing a nucleic acid encoding a Kir2.x channel in (at least part of) the stem cell derived cardiomyocytes; and
d) Allowing the stem cell derived cardiomyocytes to express the light-sensitive ion channel and the Kir2.x channel.

2. The in vitro method of claim 1 wherein the nucleic acid encoding a light-sensitive ion channel is a mRNA encoding a light-sensitive ion channel and/or wherein the nucleic acid encoding a Kir2.x channel is a mRNA encoding a Kir2.x channel.

3. The in vitro method of claim 1 wherein a subpopulation of the stem cell derived cardiomyocytes provided in step a) are treated according to step b) and wherein another subpopulation of the stem cell derived cardiomyocytes provided in step a) are treated according to step c), and wherein the subpopulations are combined after the treatments of step b) and c) and/or wherein the stem cell derived cardiomyocytes of step d) comprises stem cell derived cardiomyocytes that express Kir2.x and comprises stem cell derived cardiomyocytes that express the light-sensitive ion channel.

4. (canceled)

5. The in vitro method of claim 1 wherein the light-sensitive ion channel comprises a Channelrhodopsin, preferably comprises Channelrhodopsin1 (ChR1) or Channelrhodopsin2 (ChR2).

6. The in vitro method of claim 1 wherein the stem cell derived cardiomyocytes provided in step a) are pluripotent stem cell derived cardiomyocytes or are induced pluripotent stem cell derived cardiomyocytes.

7. The in vitro method of claim 1 wherein the stem cell derived cardiomyocytes provided in step a) are spontaneous beating stem cell derived cardiomyocytes.

8. The in vitro method of claim 1 wherein the stem cell derived cardiomyocytes provided in step a) are mammalian stem cell derived cardiomyocytes, preferably human stem cell derived cardiomyocytes.

9. The in vitro method of claim 1 wherein the nucleic acid encoding a light-sensitive ion channel encodes a fusion protein comprising the light-sensitive ion channel.

10. The in vitro method of claim 1 wherein the nucleic acid encoding a light-sensitive ion channel is a nucleic acid selected from
a) a nucleic acid having a sequence as shown in SEQ ID NO: 1.
b) a nucleic acid having a sequence that is transcribed in a sequence as shown in SEQ ID NO: 1; or
c) a nucleic acid encoding a polypeptide having at least 80% sequence identity with a polypeptide encoded by the nucleic acid of a) or b) above.

11. The in vitro method of claim 1 wherein the nucleic acid encoding a Kir 2.1 channel is a nucleic acid selected from
a) a nucleic acid having a sequence as shown in SEQ ID NO: 2.
b) a nucleic acid having a sequence that is transcribed in a sequence as shown in SEQ ID NO: 2; or
c) a nucleic acid encoding a polypeptide having at least 80% identity with a polypeptide encoded by the nucleic acid of a) or b) above.

12. (canceled)

13. The in vitro method of claim 1 wherein step b) is performed between 5 minutes and 24 hours and step c) is performed between 5 minutes and 24 hours or wherein step b) and c) is performed between 5 minutes and 24 hours.

14. The in vitro method of claim 1 wherein the method is performed using a multi-electrode array plate, preferably a multiwell multi-electrode array plate and/or wherein the cells of step a) are provided as a monolayer.

15. Stem cell derived cardiomyocytes obtainable with the method of claim 1.

16. A population of stem cell derived cardiomyocytes wherein the population of stem cell derived cardiomyocytes comprise a nucleic acid encoding a light-sensitive ion channel and comprises a nucleic acid encoding a Kir2.x channel.

17. (canceled)

18. (canceled)

19. A kit or kit-of-part comprising
a) Stem cell derived cardiomyocytes, nucleic acid encoding a light-sensitive ion channel, and nucleic acid encoding a Kir 2.x channel; or
b) Stem cell derived cardiomyocytes or population of stem cell derived cardiomyocytes produced by the method of claim 1.
c) A device, preferably a culture dish or multi well plate or assay plate comprising stem cell derived cardiomyocytes produced by the method of claim 1.

20. (canceled)

21. A method of screening a drug candidate for cardiotoxicity comprising
a) Providing stem cell derived cardiomyocytes obtainable with the method of claim 1.
b) contacting the stem cell derived cardiomyocytes of a) with a drug candidate;
c) optically activating the light-sensitive ion channel at one or multiple pacing frequencies thereby inducing contraction of the cardiomyocytes; and
d) measuring cardiomyocyte action potential, cardiomyocyte ion fluxes, cardiomyocyte field potential, impedance, contraction, movement, morphology, cardiomyocyte intracellular calcium level, velocity of conduction, or a combination thereof, thereby screening the drug candidate for cardiotoxicity or efficacy.

22. A system for screening a drug candidate for cardiotoxicity or efficacy comprising:
a) stem cell derived cardiomyocytes obtainable with the method of claim 1;
b) an optical device to activate the light sensitive ion channel at one or multiple pacing frequencies;
c) a chemical delivery device for introducing the drug candidates to be screened;
d) a sensor; optionally the sensor is an optical sensor, voltage sensor, impedance sensor, ion sensor, or a combination thereof;
e) a processor comprising executable code to process data received from the sensor; and
f) memory for storing data received from the sensor.

23. A method of optically inducing cardiomyocyte contraction comprising:
a) Providing stem cell derived cardiomyocytes obtainable with the method of claim 1; and
b) Optically activating the light-sensitive ion channel thereby inducing contraction of the cardiomyocytes.

24. A method of preventing dedifferentiation of stem cell derived cardiomyocytes obtainable with the method of claim 1 comprising optically activating the light-sensitive ion channel to induce contraction of cardiomyocytes thereby preventing dedifferentiation of stem cell derived cardiomyocytes.

25. A method of promoting maturation of stem cell derived cardiomyocytes obtainable with the method of claim 1 comprising optically activating the light-sensitive ion channel to induce contraction of the cardiomyocytes.


SEQUENCE LISTING (2 sequences)

SEQ ID NO: 1 - DNA, 933 bp, Artificial Sequence
  Synthetic
auggacuauggcggcgcuuugucugccgucggacgcgaacuuuuguucguuacuaauccugugguggugaacggguccguccuggucccugaggaucaauguuacugugccggauggauugaaucucgcggcacgaacggcgcucagaccgcgucaaauguccugcaguggcuugcagcaggauucagcauuuugcugcugauguucuaugccuaccaaaccuggaaaucuacaugcggcugggaggagaucuaugugugcgccauugaaaugguuaaggugauucucgaguucuuuuuugaguuuaagaaucccucuaugcucuaccuugccacaggacaccgggugcaguggcugcgcuaugcagaguggcugcucacuuguccugucauccuuauccgccugagcaaccucaccggccugagcaacgacuacagcaggagaaccaugggacuccuugucucagacaucgggacuaucguguggggggcuaccagcgccauggcaaccggcuauguuaaagucaucuucuuuugucuuggauugugcuauggcgcgaacacauuuuuucacgccgccaaagcauauaucgaggguuaucauacugugccaaagggucggugccgccaggucgugaccggcauggcauggcuguuuuucgugagcugggguauguucccaauucucuucauuuuggggcccgaagguuuuggcguccugagcgucuauggcuccaccguaggucacacgauuauugaucugaugaguaaaaauuguuggggguuguugggacacuaccugcgcguccugauccacgagcacauauugauucacggagauauccgcaaaaccaccaaacugaacaucggcggaacggagaucgaggucgagacucucgucgaagacgaagccgaggccggagccgugccauaa

SEQ ID NO: 2 - DNA, 1284 bp, Artificial Sequence
  Synthetic
augggcagugugcgaaccaaucgcuacagcaucgucucuucagaagaagacgguaugaaguuggccaccauggcaguugcaaauggcuuugggaacgggaagaguaaaguccacacccgacaacagugcaggagccgcuuugugaagaaagauggccacuguaauguucaguucaucaaugugggugagaaggggcaacgguaccucgcagacaucuucaccacguguguggacauucgcuggcgguggaugcugguuaucuucugccuggcuuucguccugucauggcuguuuuuuggcuguguguuuugguugauagcucugcuccauggggaccuggaugcauccaaagagggcaaagcuuguguguccgaggucaacagcuucacggcugccuuccucuucuccauugagacccagacaaccauaggcuaugguuucagaugugucacggaugaaugcccaauugcuguuuucauggugguguuccagucaaucgugggcugcaucaucgaugcuuucaucauuggcgcagucauggccaagauggcaaagccaaagaagagaaacgagacucuugucuucagucacaaugccgugauugccaugagagacggcaagcuguguuugauguggcgagugggcaaucuucggaaaagccacuugguggaagcucauguucgugcacagcuccucaaauccagaauuacuucugaaggggaguauaucccucuggaucaaauagacaucaauguuggguuugacaguggaaucgaucguauauuucugguguccccaaucacuauaguccaugaaauagaugaagacaguccuuuauaugauuugaguaaacaggacauugacaacgcagacuuugaaaucguggucauacuggaaggcaugguggaagccacugccaugacgacacagugccguagcucuuaucuagcaaaugaaauccuguggggccaccgcuaugagccugugcucuuugaagagaagcacuacuacaaaguggacuauuccagguuccacaaaacuuacgaaguccccaacacuccccuuuguagugccagagacuuagcagaaaagaaauauauccucucaaaugcaaauucauuuugcuaugaaaaugaaguugcccucacaagcaaagaggaagacgacagugaaaauggaguuccagaaagcacuaguacggacacgcccccugacauagaccuucacaaccaggcaaguguaccucuagagcccaggcccuuacggcgagagucggagauauga